(19) United States
(12) Patent Application Publication
(10) Pub. No.: US 2007/0232624 A1
(43) Pub. Date: Oct. 4, 2007

(54) USE OF PALIPERIDONE FOR THE TREATMENT OF SLEEP DISTURBANCES AND/OR EXCESSIVE DAYTIME SLEEPINESS IN PSYCHIATRIC PATIENTS

(76) Inventors: Joseph M. Palumbo, Saint Davids, CA (US); Marie-Henriette Eerdekens, Dilsen-Stokkern (BE); Michelle Kramer, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/534,632

(22) Filed: Sep. 22, 2006

Related U.S. Application Data
(60) Provisional application No. 60/788,764, filed on Apr. 3, 2006.

(51) Int. Cl. A61K 31/505

(52) U.S. Cl. 514/259.4

(57) ABSTRACT

The present invention provides a pharmaceutical composition, and provides for the use thereof for the treatment of excessive daytime sleepiness and/or a sleep disturbance in a psychiatric patient in need thereof. The pharmaceutical composition comprises a therapeutically effective amount of paliperidone, its pharmaceutically acceptable acid addition salts, enantiomeric forms, and esters thereof, together with a pharmaceutical carrier, and provides a relatively low plasma concentration variation of paliperidone to the psychiatric patient in need of treatment. The present invention also provides for the use of a pharmaceutical composition as defined above, in the preparation of a medicament for the treatment of excessive daytime sleepiness and/or a sleep disturbance in a psychiatric patient in need thereof.

This application claims priority and benefit of provisional application 60/788,764 filed Apr. 3, 2006, which is hereby incorporated by reference herein.

### FIELD OF THE INVENTION

This invention relates to the use of paliperidone for the treatment of persistent sleep disturbances and/or excessive daytime sleepiness in psychiatric patients.

### BACKGROUND OF THE INVENTION

Patients with mental illness often have sleep disturbances including insomnia, and excessive sleeping. Sleep disturbances are common in patients with schizophrenia, depression and bipolar disorders. For example, patients with schizophrenia appear to have longer sleep latency, a higher number of arousals during sleep and increased periods of wakefulness after sleep onset compared with those in nonpsychiatric control groups. (Tandon et al. and Miller et al.). Patients being treated for psychotic symptoms may also suffer from excessive daytime sleepiness or drowsiness associated with both sleep disturbance and sedation caused by antipsychotic medications. Additionally, the excessive sedation caused by some antipsychotic medications can result in impaired cognitive functions and other symptoms that interfere with successful treatment of the patient. More importantly, sedation can impair a patient's quality of life and efforts to lead a normal life.

### SUMMARY OF THE INVENTION

We have discovered that, by treating psychiatric patients who suffer from excessive daytime sleepiness and/or a sleep disturbance with a pharmaceutically effective amount of paliperidone, its pharmaceutically acceptable acid addition salts, enantiomeric forms, and esters, delivered at a relatively constant plasma level, these patients will have significantly reduced daytime drowsiness and/or sleep disturbances.

In one embodiment, the present invention provides for the use of a pharmaceutical composition comprising a therapeutically effective amount of paliperidone, its pharmaceutically acceptable acid addition salts, enantiomeric forms, or esters thereof, together with a pharmaceutically acceptable carrier, for the treatment of excessive daytime sleepiness and/or a sleep disturbance in a psychiatric patient in need thereof, the pharmaceutical composition providing a relatively low plasma concentration variation of paliperidone to the psychiatric patient in need of treatment.

In yet another embodiment, the present invention provides for the use of a pharmaceutical composition comprising a therapeutically effective amount of paliperidone, its pharmaceutically acceptable acid addition salts, enantiomeric forms, or esters thereof, together with a pharmaceutically acceptable carrier, in the preparation of a medicament for the treatment of excessive daytime sleepiness and/or a sleep disturbance in a psychiatric patient in need thereof, the pharmaceutical composition providing a relatively low plasma concentration variation of paliperidone to the psychiatric patient in need of treatment.

In another embodiment, the invention provides a pharmaceutical composition comprising a therapeutically effective amount of paliperidone, its pharmaceutically acceptable acid addition salts, enantiomeric forms, or esters thereof, together with a pharmaceutically acceptable carrier, for the treatment of excessive daytime sleepiness and/or a sleep disturbance in a psychiatric patient in need thereof, the pharmaceutical composition providing a relatively low plasma concentration variation of paliperidone to the psychiatric patient in need of treatment.

In another embodiment, the invention provides for the use of a pharmaceutical composition comprising a therapeutically effective amount of paliperidone, its pharmaceutically acceptable acid addition salts, enantiomeric forms, or esters thereof, together with a pharmaceutically acceptable carrier, for improving symptoms of schizophrenia in a psychiatric patient in need thereof, wherein the symptoms are selected from the group consisting of sleep disturbances and excessive daytime sleepiness, the pharmaceutical composition providing a relatively low plasma concentration variation of paliperidone to the psychiatric patient in need of treatment.

In another embodiment, the invention provides for the use of a pharmaceutical composition comprising a therapeutically effective amount of paliperidone, its pharmaceutically acceptable acid addition salts, enantiomeric forms, or esters thereof, together with a pharmaceutically acceptable carrier, in the manufacture of a medicament for improving symptoms of schizophrenia in a psychiatric patient in need thereof, wherein the symptoms are selected from the group consisting of sleep disturbances and excessive daytime sleepiness, the pharmaceutical composition providing a relatively low plasma concentration variation of paliperidone to the psychiatric patient in need of treatment.

In still another embodiment, the invention provides a pharmaceutical composition comprising a therapeutically effective amount of paliperidone, its pharmaceutically acceptable acid addition salts, enantiomeric forms, or esters thereof, together with a pharmaceutically acceptable carrier, for improving symptoms of schizophrenia in a psychiatric patient in need thereof, wherein the symptoms are selected from the group consisting of sleep disturbances and excessive daytime sleepiness, the pharmaceutical composition providing a relatively low plasma concentration variation of paliperidone to the psychiatric patient in need of treatment.

These and other objects and advantages of the present invention may be appreciated from a review of the present applications.

### BRIEF DESCRIPTION OF THE FIGURE

FIG. 1 provides a graphical presentation of a relatively constant plasma release profile of paliperidone from a controlled release dosage form such as an OROS dosage form as compared to an immediate release form of paliperidone. The data points shown for day 1 are measured at specific time intervals as is described in Example 4. For days 2 through 6, data points were measured at 2 and 22 hours post administration and constitute an average of the results from each group at each time. The data points for paliperidone shown between days 6 and 7 show multiple time points during the day, illustrating the intraday variance in plasma level concentration of paliperidone.

### DETAILED DESCRIPTION

Sleep disturbances and excessive daytime drowsiness occur in patients suffering from many mental illnesses. These subjects spend an excessive amount of time in a sleep state or unable to maintain a satisfactory degree of wakefulness during a period of the day when wakefulness is required or desired.

As used herein the term “sleep disturbance” shall mean a condition in which an individual has longer sleep latency, a higher number of arousals during sleep, and increased periods of wakefulness after sleep onset (e.g. hypersomnia, dyssomnia and parasomnias). These disorders may be diagnosed using conventional methods such as are commonly employed in a sleep laboratory (e.g. polysomnography).

As used herein the term “Excessive Daytime Sleepiness” (EDS) shall be used interchangeably with the term “pathological somnolence” and shall mean a condition in which an individual feels very drowsy during the day and finds it difficult to resist the urge to fall asleep, whether or not the individual has gotten enough nighttime sleep. Excessive daytime sleepiness (EDS) is defined as sleepiness occurring in a situation when an individual would be expected to be awake and alert. Clinically the symptoms of EDS can be quantified and measured in a variety of ways, including but not limited to; the Multiple Sleep Latency Test (MSLT) (See Carskadon M A and Dement W C, Sleep 1982;5 Suppl 2:S67-72), the Maintenance of Wakefulness Test (MWT) (See, Mitler M M, et al. Electroencephalogr Clin Neurophysiol, 1982;53(6):658-61) or the Stanford Sleepiness Scale (SSS) (See, Hoddes E et al., Psychophysiology, 1973;10(4):431-6) (See also, Arand D et al. Sleep, 2005;28(1):123-144). The causes of EDS are multiple and the use of the term EDS herein is not intended to imply any particular cause or etiology. People with EDS frequently doze, nap, or fall asleep in situations where they need or want to be fully awake and alert. The diagnosis can be made when the symptoms of EDS interfere significantly with a person's ability to concentrate and perform daily tasks and routines such as work, family responsibilities, driving a car or operating other hazardous machinery or general quality of life.

The term “psychiatric patient” as used herein, refers to a human, who has been the object of treatment, or experiment for a “mental disorder”, and the term “mental disorder” also encompasses mental illnesses and refers to those provided in the Diagnostic and Statistical Manual (DSM III or IV), American Psychological Association (APA). These mental disorders include schizophrenia; bipolar disorder or other disease states in which psychosis, aggressive behavior, anxiety or depression is evidenced. Schizophrenia refers to conditions characterized as schizophrenia, schizoaffective disorder and schizophreniform disorders, in DSM-IV-TR such as category 295.xx. Bipolar Disorder refers to a conditions characterized as a Bipolar Disorder, in DSM-IV-TR such as category 296.xx including Bipolar I and Bipolar Disorder II. The DSM-IV-R was prepared by the Task Force on Nomenclature and Statistics of the American Psychiatric Association, and provides clear descriptions of diagnostic categories. Pathologic psychological conditions, which are psychoses or may be associated with psychotic features include, but are not limited to the following disorders that have been characterized in the DSM-IV-TR. Diagnostic and Statistical Manual of Mental Disorders, Revised, 3rd Ed. (1994). The numbers in parenthesis refer to the DSM-IV-TR categories. The skilled artisan will recognize that there are alternative nomenclatures, nosologies, and classification systems for pathologic psychological conditions and that these systems evolve with medical scientific progress.

Examples of pathologic psychological conditions which may be treated include, but are not limited to,. Mild Mental Retardation (317), Moderate Mental Retardation (318.0), Severe Mental Retardation (318.1), Profound Mental Retardation (318.2), Mental Retardation Severity Unspecified (319), Autistic Disorders (299.00), Rett's Disorder (299.80), Childhood Disintegrative Disorders (299.10), Asperger's Disorder (299.80), Pervasive Developmental Disorder Not Otherwise Specified (299.80), Attention-Deficit/Hyperactivity Disorder Combined Type (314.01), Attention-Deficit/Hyperactivity Disorder Predominately Inattentive Type (314.00), Attention-Deficit/Hyperactivity Disorder Predominately Hyperactive-Impulsive Type (314.01), Attention-Deficit/Hyperactivity Disorder NOS (314.9) Conduct Disorder (Childhood-Onset and Adolescent Type 312.8) Oppositional Defiant Disorder (313.81), Disruptive Behavior Disorder Not Otherwise Specified (312.9), Solitary Aggressive Type (312.00), Conduct Disorder, Undifferentiated Type (312.90), Tourette's Disorder (307.23), Chronic Motor Or Vocal Tic Disorder (307.22), Transient Tic Disorder (307.21), Tic Disorder NOS (307.20), Alcohol Intoxication Delirium (291.0), Alcohol Withdrawal Delirium (291.0), Alcohol-Induced Persisting Dementia (291.2), Alcohol-Induced Psychotic Disorder with Delusions (291.5), Alcohol-Induced Psychotic Disorder with Hallucinations (291.3), Amphetamine or Similarly Acting Sympathomimetic Intoxication (292.89), Amphetamine or Similarly Acting Sympathomimetic Delirium (292.81), Amphetamine or Similarly Acting Sympathomimetic Induced Psychotic with Delusional (292.11), Amphetamine or Similarly Acting Sympathomimetic Induced Psychotic with Hallucinations (292.12), Cannabis-Induced Psychotic Disorder with Delusions (292.11), Cannabis-Induced Psychotic Disorder with Hallucinations (292.12), Cocaine Intoxication (292.89), Cocaine Intoxication Delirium (292.81), Cocaine-Induced Psychotic Disorder with Delusions (292.11), Cocaine-Induced Psychotic Disorder with Hallucinations (292.12), Halluciogen Intoxication (292.89), Hallucinogen Intoxication Delirium (292.81), Hallucinogen-Induced Psychotic disorder with Delusions (292.11), Hallucinogen-Induced Psychotic disorder with Delusions (292.12), Hallucinogen-Induced Mood Disorder (292.84), Hallucinogen-Induced Anxiety Disorder (292.89), Hallucinogen-Related Disorder Not Otherwise Specified (292.9), Inhalant Intoxication (292.89), Inhalant Intoxication Delirium (292.81), Inhalant-Induced Persisting Dementia (292.82), Inhalant-Induced Psychotic Disorder with Delusions (292.11), Inhalant-Induced Psychotic with Hallucinations (292.12), Inhalant-Induced Mood Disorder (292.89), Inhalant-Induced Anxiety Disorder (292.89), Inhalant-Related Disorder Not Otherwise Specified (292.9), Opioid Intoxication Delirium (292.81), Opioid-Induced Psychotic Disorder with Delusions (292.11), Opioid Intoxication Delirium (292.81), Opioid-Induced Psychotic Disorder with Hallucinations (292.12), Opioid-Induced Mood Disorder (292.84), Phencyclidine (PCP) or Similarly Acting Arylcyclohexylamine Intoxication (292.89), Phencyclidine (PCP) or Similarly Acting Arylcyclohexylamine Intoxication Delirium (292.81), Phencyclidine (PCP) or Similarly Acting Arylcyclohexylamine Induced Psychotic Disorder with Delusions (292.11), Phencyclidine (PCP) or Similarly Acting Arylcyclohexylamine Induced Psychotic Disorder with Hallucinations (292.12), Phencyclidine (PCP) or Similarly Acting Arylcyclohexylamine Mood Disorder (292.84), Phencyclidine (PCP) or Similarly Acting Arylcyclohexylamine Induced Anxiety Disorder (292.89), Phencyclidine (PCP) or Similarly Acting Arylcyclohexylamine Related Disorder Not Otherwise Specified (292.9), Sedative, Hypnotic or Anxiolytic Intoxication (292.89), Sedation, Hypnotic or Anxiolytic Intoxication Delirium (292.81), Sedation, Hypnotic or Anxiolytic Withdrawal Delirium (292.81), Sedation, Hypnotic or Anxiolytic Induced Persisting Dementia (292.82), Sedation, Hypnotic or Anxiolytic-Induced Psychotic Disorder with Delusions (292.11), Sedation, Hypnotic or Anxiolytic-Induced Psychotic Disorder with Hallucinations (292.12), Sedation, Hypnotic or Anxiolytic-Induced Mood Disorder (292.84), Sedation, Hypnotic or Anxiolytic-Induced Anxiety Disorder (292.89), Other (or Unknown) Substance Intoxication (292.89), Other (or Unknown) Substance-Induced Delirium (292.81), Other (or Unknown) Substance-Induced Persisting Dementia (292.82), Other (or Unknown) Substance-Induced Psychotic Disorder with Delusions (292.11), Other (or Unknown) Substance-Induced Psychotic Disorder with Hallucinations (292.12), Other (or Unknown) Substance-Induced Mood Disorder (292.84), Other (or Unknown) Substance-Induced Anxiety Disorder (292.89), Other (or Unknown) Substance Disorder Not Otherwise Specified (292.9), Obsessive Compulsive Disorder (300.3), Post-traumatic Stress Disorder (309.81), Generalized Anxiety Disorder (300.02), Anxiety Disorder Not Otherwise Specified (300.00), Body Dysmorphic Disorder (300.7), Hypochondriasis (or Hypochondriacal Neurosis) (300.7), Somatization Disorder (300.81), Undifferentiated Somatoform Disorder (300.81), Somatoform Disorder Not Otherwise Specified (300.81), Intermittent Explosive Disorder (312.34), Kleptomania (312.32), Pathological Gambling (312.31), Pyromania (312.33), Trichotillomania (312.39), and Impulse Control Disorder NOS (312.30), Schizophrenia, Paranoid Type, (295.30), Schizophrenia, Disorganized (295.10), Schizophrenia, Catatonic Type, (295.20), Schizophrenia, Undifferentiated Type (295.90), Schizophrenia, Residual Type (295.60), Schizophreniform Disorder (295.40), Schizoaffective Disorder (295.70), Delusional Disorder (297.1), Brief Psychotic Disorder (298.8), Shared Psychotic Disorder (297.3), Psychotic Disorder Due to a General Medical Condition with Delusions (293.81), Psychotic Disorder Due to a General Medical Condition with Hallucinations (293.82), Psychotic Disorders Not Otherwise Specified (298.9), Major Depression, Single Episode, Severe, without Psychotic Features (296.23), Major Depression, Recurrent, Severe, without Psychotic Features (296.33), Bipolar Disorder, Mixed, Severe, without Psychotic Features (296.63), Bipolar Disorder, Mixed, Severe, with Psychotic Features (296.64), Bipolar Disorder, Manic, Severe, without Psychotic Features (296.43), Bipolar Disorder, Manic, Severe, with Psychotic Features (296.44), Bipolar Disorder, Depressed, Severe, without Psychotic Features (296.53), Bipolar Disorder, Depressed, Severe, with Psychotic Features (296.54), Bipolar II Disorder (296.89), Bipolar Disorder Not Otherwise Specified (296.80), Personality Disorders, Paranoid (301.0), Personality Disorders, Schizoid (301.20), Personality Disorders, Schizotypal (301.22), Personality Disorders, Antisocial (301.7), and Personality Disorders, Borderline (301.83).

The term “a patient in need of treatment” as used herein will refer to any subject or psychiatric patient who currently has or may develop any of the above syndromes or disorders, including any condition or disorder in which the subject spends an excessive amount of time in a sleep state or unable to maintain a satisfactory degree of wakefulness during a period of the day when wakefulness is required or desired or has EDS.

Paliperidone, including its pharmaceutically acceptable acid addition salts, enantiomeric forms and esters may be administered for the practice of the present invention. Paliperidone is well known in the art and is described in U.S. Pat. No. 5,158,952, which is hereby incorporated by reference herein.

As noted in U.S. Pat. No. 5,158,952, paliperidone has basic properties and, consequently, this compound may be converted to its therapeutically active non-toxic acid addition salt forms by treatment with appropriate acids, such as, for example, inorganic acids, such as hydrohalic acid, e.g. hydrochloric, hydrobromic acid and the like, sulfuric acid, nitric acid, phosphoric acid and the like; or organic acids, such as, for example, acetic, propanoic, hydroxyacetic, 2-hydroxypropanoic, 2-oxopropanoic, ethanedioic, propanedioic, butanedioic, (Z)-2-butenedioic, (E)-2-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids. Conversely the salt form can be converted into the free base form by treatment with alkali. The term acid addition salt as used hereinabove also comprises the solvates which such compounds are able to form and said solvates are meant to be included within the scope of the present invention. Examples of such solvates are e.g., the hydrates, alcoholates and the like.

Esters of Paliperidone are known in the art and are described in U.S. Pat. No. 5,254,556, which is hereby incorporated by reference. Esters of paliperidone include octanoic acid, decanoic acid, dodecanic acid, tetradecanoic acid or hexadecanoic acid (palmitic acid). The currently preferred ester of paliperidone is paliperidone palmitate.

Paliperidone may be formulated with pharmaceutical excipients into a variety of dosage forms as described in U.S. Pat. No. 5,158,952. Paliperidone will in one embodiment of the present invention be provided in an oral dosage form. Suitable oral dosage forms include but are not limited to controlled release or extended release dosage forms. Currently preferred are once daily extended release dosage forms. Most preferred are extended release OROS oral dosage forms which are well known in the art. Examples of oral dosage forms of paliperidone are described in US 20040092534, US 20050208132 and US 20050232995, all of which are hereby incorporated by reference herein.

Paliperidone palmitate, including its pharmaceutically acceptable acid addition salts, and stereoisomeric forms, is also well known in the art and may also be formulated with pharmaceutical excipients into a variety of dosage forms as described in U.S. Pat. No. 5,254,556, which is hereby incorporated by reference herein. Currently, it is preferred to administer paliperidone palmitate in a depot.

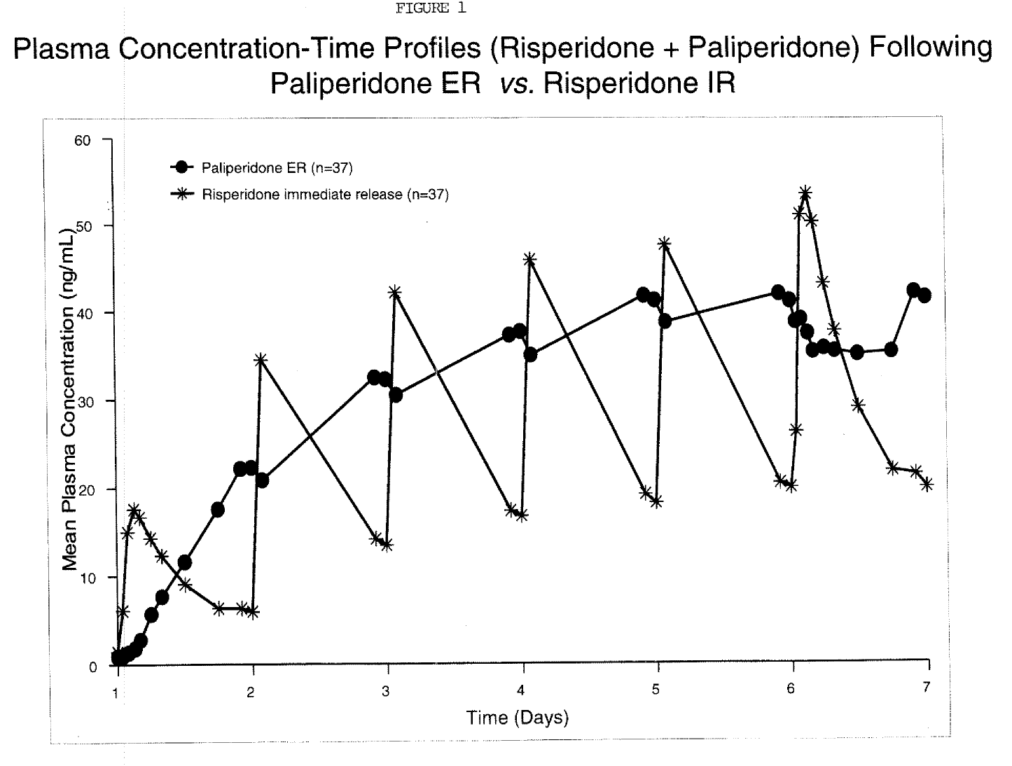

For the practice of this invention paliperidone should be delivered with a dosage form that will provide low plasma concentration variation (peak to trough) for the patient receiving treatment to reduce excessive daytime sleepiness and/or sleep disturbance. Low plasma concentration variation means that the intradose variation in plasma concentration (Cmax-Cmin)/Cmax has been minimized. As illustrated in FIG. 1 a low plasma concentration variation is achieved by the dosage form being tested (specifically between day 6 and 7). The intradose variation in plasma concentration from the maximum plasma concentration (C max) to the minimum plasma concentration (Cmin) when minimized reduces excessive daytime sleepiness and/or sleep disturbance. In one embodiment of the invention, the reduction in plasma concentration from Cmax to Cmin will be less than about 35 percent, preferably less than about 30 percent, more preferably from about 10 percent to about 30 percent, most preferably from about 15 percent to about 25 percent. These ranges are calculated as (Cmax-Cmin)/Cmax from the administration of one dosage form to the next. Preferably the dosing will be once per day.

For the avoidance of doubt “intradose” or “intradose period” refers to the period between the initiation of drug release by a dosage form from its administration until the administration of the next dose of said dosage form in a dosing regiment. Generally, this period will be the period from the administration of one dose to the administration of another dose, except where a dosage form does not release drug immediately upon administration. In this case, the period of delay of release of the drug will have to be considered. This adjustment should be considered in establishing Cmin.

Additionally, FIG. 1 illustrates a flat plasma curve. “Flat plasma curve” means a plasma concentration curve that reaches and maintains a substantially constant value after a defined period of time following administration of a dosage form according to the invention. A steady state wherein the absolute values of Cmax and Cmin are relatively constant from day to day is achieved after about 6 days.

“C” means the concentration of drug in blood plasma, or serum, of a subject, generally expressed as mass per unit volume, typically nanograms per milliliter. For convenience, this concentration may be referred to herein as “drug plasma concentration”, “plasma drug concentration” or “plasma concentration”. The plasma drug concentration at any time following drug administration is referenced as Ctime, as in C9 h or C24 h, etc. A maximum plasma concentration obtained following administration of a dosage form obtained directly from the experimental data without interpolation is referred to as Cmax. A minimum plasma concentration obtained following administration of a dosage form obtained directly from experimental data without interpolation is referred Cmin.

Persons of skill in the art will appreciate that blood plasma drug concentrations obtained in individual subjects will vary due to inter-patient variability in the many parameters affecting drug absorption, distribution, metabolism and excretion. For this reason, unless otherwise indicated, when a drug plasma concentration is listed, the value listed is the calculated mean value based on values obtained from a groups of subjects tested.

Those of ordinary skill in the art will appreciate that paliperidone, its salts, enantiomers, and esters, can be administered for all the known uses of risperidone. For example paliperidone can be used to treat schizophrenia, schizoaffective disorders, bipolar mania or other disease states in which psychosis, aggressive behavior, anxiety or depression is evidenced (e.g. psychosis associated with dementia, post traumatic stress syndrome, etc.).

As used herein the term “subject”, refers to an animal, preferably a mammal, and most preferably a human, who has been the object of treatment, observation or experiment.

The term “therapeutically effective amount” as used herein, means that amount of active compound or pharmaceutical agent that elicits the biological or medicinal response in human that is being sought by a researcher, medical doctor or other clinician, which includes alleviation of the symptoms of the disease or disorder being treated.

Those of skill in the treatment of diseases could easily determine the effective amount of paliperidone to administer for the treatment of the diseases listed above. In general it is contemplated that an effective amount would be from about 0.01 mg/kg to about 2 mg/kg body weight. In one embodiment of the present invention, paliperidone is administered in an oral dosage form to a subject once a day. The mg of compound delivered in such a dosage form to the patient may be from 0.25 to about 20 mg (e.g. 0.25 mg, 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, and 20 mg) per oral dosage form.

### EXAMPLE 1

### Paliperidone Capsule Shaped Tablet, Trilayer 2 mg System

A dosage form adapted, designed and shaped as an osmotic drug delivery device was manufactured as follows: 120 g of paliperidone, 7325 g of polyethylene oxide with average molecular weight of 200,000, and 2000 g of sodium chloride, USP were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a second drug compartment composition was prepared as follows: 280 g of paliperidone and 9165 g of polyethylene oxide with average molecular weight of 200,000 were added to a fluid bed granulator bowl. Next a binder solution was prepared by dissolving 400 g of hydroxypropylmethyl cellulose identified as 2910 having an average viscosity of 5 cps in 7,600 g of water. The dry materials were fluid bed granulated by spraying with 4,000 g of binder solution. Next, the wet granulation was dried in the granulator to an acceptable moisture content, and sized using by passing through a 7-mesh screen. Next, the granulation was transferred to a blender and mixed with 5 g of butylated hydroxytoluene as an antioxidant and lubricated with 50 g of stearic acid.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the paliperidone drug compositions for the first and the second compartments and the push composition were compressed into trilayer tablets. First, 50 mg of the paliperidone compartment one composition was added to the die cavity and pre-compressed, then 50 mg of the paliperidone compartment two composition was added to the die cavity and pre-compressed, then 100 mg of the push composition was added and the layers were pressed into a 3/16″ diameter longitudinal, deep concave, trilayer arrangement.

The trilayered arrangements were coated with a subcoat laminate. The wall forming composition comprised 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an 8% solids solution. The wall-forming composition was sprayed onto and around the bilayered arrangements in a pan coater until approximately 20 mg of laminate was applied to each tablet.

The trilayered arrangements were coated with a semi-permeable wall. The wall forming composition comprised 99% cellulose acetate having a 39.8% acetyl content and 1% polyethylene glycol comprising a 3.350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 40 mg of membrane was applied to each tablet.

Next, two 25 mil (0.6 mm) exit passageways were laser drilled through the semi-permeable wall to connect the drug layer with the exterior of the dosage system. The residual solvent was removed by drying for 144 hours at 45° C. and 45% humidity. After drilling, the osmotic systems were dried for 4 hours at 45° C. to remove excess moisture.

The dosage form produced by this manufacture was designed to deliver 2 mg of paliperidone in an ascending delivery pattern from two drug-containing cores. The first core contained 1.2% paliperidone, 73.25% polyethylene oxide possessing a 200,000 molecular weight, 20% sodium chloride, USP, 5% hydroxypropylmethyl cellulose having an average viscosity of 5 cps, 0.05% butylated hydroxytoluene, and 0.5% stearic acid. The second drug core contained 2.8% paliperidone, 91.65% polyethylene oxide possessing a 200,000 molecular weight, 5% hydroxypropylmethyl cellulose having an average viscosity of 5 cps, 0.05% butylated hydroxytoluene, and 0.5% stearic acid. The push composition comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The semi permeable wall was comprised of 99% cellulose acetate of 39.8% acetyl content and 1% polyethylene glycol. The dosage form comprised two passageways, 25 mils (0.6 mm) on the center of the drug side.

### EXAMPLE 2

### Paliperidone Capsule Shaped Tablet, Trilayer 3 mg System

A dosage form adapted, designed and shaped as an osmotic drug delivery device is manufactured as follows: 2.246 kg of paliperidone, 87.2 kg of polyethylene oxide with average molecular weight of 200,000 and 24 kg of sodium chloride, USP are added to a fluid bed granulator bowl. Paliperidone amount includes a 4% manufacturing excess to compensate for losses during processing. Next, a binder solution is prepared by dissolving 7.2 kg of polyvinylpyrrolidone identified as K29-32 having an average molecular weight of 40,000 in 52.8 kg of water. The dry materials are fluid bed granulated by spraying with 50 kg of binder solution. Next, the wet granulation is dried in the granulator to the acceptable moisture content. Then, the dried granulation is sized using a Fluid Air mill with a 7-mesh screen. The granulation is transferred to a tote tumbler, mixed with 60 g of butylated hydroxytoluene and lubricated with 600 g stearic acid.

Next, a second drug compartment composition is prepared as follows: 5.242 kg of paliperidone, 0.06 kg of yellow ferric oxide and 108.2 kg of polyethylene oxide with average molecular weight of 200,000 are added to a fluid bed granulator bowl. Paliperidone amount includes a 4% manufacturing excess to compensate for losses during processing. Next, a binder solution is prepared by dissolving 7.2 kg of polyvinylpyrrolidone identified as K29-32 having an average molecular weight of 40,000 in 52.8 kg of water. The dry materials are fluid bed granulated by spraying with 50 kg of binder solution. Next, the wet granulation is dried in the granulator to the acceptable moisture content. Then, the dried granulation is sized using a Fluid Air mill with a 7-mesh screen. The granulation is transferred to a tote tumbler, mixed with 60 g of butylated hydroxytoluene and lubricated with 600 g stearic acid.

Next, a push composition is prepared as follows: first, a binder solution is prepared. 30 kg of polyvinylpyrrolidone identified as K29-32 having an average molecular weight of 40,000 is dissolved in 200.8 kg of water. Then, 100 kg of sodium chloride and 5 kg of red ferric oxide are sized using a Quadro Comil with a 21-mesh screen. Then, the screened materials and 368.50 kg of Polyethylene oxide (approximately 7,000,000 molecular weight) are added to a fluid bed granulator bowl. The dry materials are fluidized and mixed while 192 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 240 g of butylated hydroxytoluene and lubricated with 1200 g stearic acid.

Next, the paliperidone drug compositions for the first and the second compartments and the push composition are compressed into trilayer tablets. First, 50 mg of the paliperidone compartment one composition is added to the die cavity and pre-compressed, then 50 mg of the paliperidone compartment two composition is added to the die cavity and pre-compressed, then 100 mg of the push composition is added and the layers are pressed into a 3/16″ diameter longitudinal, deep concave, trilayer arrangement.

The trilayered arrangements are coated with a subcoat laminate. The wall forming composition comprises 95% hydroxyethylcellulose and 5% of polyethylene glycol comprising a 3.350 viscosity-average molecular weight. The wall-forming composition is dissolved in water, to make an 8% solids solution. The wall-forming composition is sprayed onto and around the bilayered arrangements in a pan coater until approximately 10 mg of laminate is applied to each tablet.

The trilayered arrangements are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co solvent to make a 5% solids solution. The wall-forming composition is sprayed onto and around the bilayered arrangements in a pan coater until approximately 45 mg of membrane is applied to each tablet.

Next, two 25 mil (0.64 mm) exit passageways are laser drilled through the semi-permeable wall to connect the drug layer with the exterior of the dosage system. The residual solvent is removed by drying for 144 hours at 45° C. and 45% humidity followed with approximately 1 hour at 45° C. to remove excess moisture.

The dosage form produced by this manufacture is designed to deliver 3 mg of paliperidone in an ascending delivery pattern from two drug-containing cores.

### EXAMPLE 3

### Paliperidone Capsule Shaped Tablet, Trilayer 9 mg System

A dosage form adapted, designed and shaped as an osmotic drug delivery device is manufactured as follows:6.64 kg of paliperidone, 82.86 kg of polyethylene oxide with average molecular weight of 200,000 and 24 kg of sodium chloride, USP are added to a fluid bed granulator bowl. Paliperidone amount includes a 2.5% manufacturing excess to compensate for losses during processing. Next, a binder solution is prepared by dissolving 7.2 kg of polyvinylpyrrolidone identified as K29-32 having an average molecular weight of 40,000 in 52.8 kg of water. The dry materials are fluid bed granulated by spraying with 50 kg of binder solution. Next, the wet granulation is dried in the granulator to the acceptable moisture content. Then, the dried granulation is sized using a Fluid Air mill with a 7-mesh screen. The granulation is transferred to a tote tumbler, mixed with 60 g of butylated hydroxytoluene and lubricated with 600 g stearic acid.

Next, a second drug compartment composition is prepared as follows: 15.50 kg of paliperidone, 0.018 kg of black iron oxide, and 98.20 kg of polyethylene oxide with average molecular weight of 200,000 are added to a fluid bed granulator bowl. Paliperidone amount includes a 2.5% manufacturing excess to compensate for losses during processing. Next, a binder solution is prepared by dissolving 7.2 kg of polyvinylpyrrolidone identified as K29-32 having an average molecular weight of 40,000 in 52.8 kg of water. The dry materials are fluid bed granulated by spraying with 50 kg of binder solution. Next, the wet granulation is dried in the granulator to the acceptable moisture content. Then, the dried granulation is sized using a Fluid Air mill with a 7-mesh screen. The granulation is transferred to a tote tumbler, mixed with 60 g of butylated hydroxytoluene and lubricated with 600 g stearic acid.

Next, a push composition is prepared as follows: first, a binder solution is prepared. 30 kg of polyvinylpyrrolidone identified as K29-32 having an average molecular weight of 40,000 is dissolved in 200.8 kg of water. Then, 100 kg of sodium chloride and 5 kg of red ferric oxide are sized using a Quadro Comil with a 21-mesh screen. Then, the screened materials and 368.50 kg of Polyethylene oxide (approximately 7,000,000 molecular weight) are added to a fluid bed granulator bowl. The dry materials are fluidized and mixed while 192 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 240 g of butylated hydroxytoluene and lubricated with 1200 g stearic acid.

Next, the paliperidone drug compositions for the first and the second compartments and the push composition are compressed into trilayer tablets. First, 50 mg of the paliperidone compartment one composition is added to the die cavity and pre-compressed, then 50 mg of the paliperidone compartment two composition is added to the die cavity and pre-compressed, then 100 mg of the push composition is added and the layers are pressed into a 3/16″ diameter longitudinal, deep concave, trilayer arrangement.

The trilayered arrangements are coated with a subcoat laminate. The wall forming composition comprises 95% hydroxyethylcellulose and 5% of polyethylene glycol comprising a 3.350 viscosity-average molecular weight. The wall-forming composition is dissolved in water, to make an 8% solids solution. The wall-forming composition is sprayed onto and around the bilayered arrangements in a pan coater until approximately 10 mg of laminate is applied to each tablet.

The trilayered arrangements are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co solvent to make a 5% solids solution. The wall-forming composition is sprayed onto and around the bilayered arrangements in a pan coater until approximately 45 mg of membrane is applied to each tablet.

Next, two 25 mil (0.64 mm) exit passageways are laser drilled through the semi-permeable wall to connect the drug layer with the exterior of the dosage system. The residual solvent is removed by drying for 144 hours at 45° C. and 45% humidity followed with approximate 1 hour at 45° C. to remove excess moisture.

The dosage form produced by this manufacture is designed to deliver 9 mg of paliperidone in an ascending delivery pattern from two drug-containing cores.

### EXAMPLE 4

### Determination of Plasma Profile of Paliperidone OROS formulation

### Dosage and Administration

RISPERIDAL® (risperidone IR) brand risperidone was administered to subjects (in these examples the subjects are human patients). The initial dose of risperidone was 2 mg/day and titrated to 4 mg/day on Day 2. Because the bioavailability of ER OROS paliperidone is approximately 30% that of an oral solution of paliperidone, 12 mg ER OROS paliperidone dose was selected to be administered as an equivalent dose with respect to plasma levels to 4 mg IR risperidone. The ER OROS paliperidone was provided as 2 mg tablets made substantially as described in Example 1.

Subjects in each group were given a once-daily oral dose, consisting of six (6) 2 mg capsules, every morning during the washout and double-blind treatment periods. Study medication was administered sitting in an upright position with 200 mL of water immediately after a standardized breakfast eaten between approximately 8:00 a.m. and 10:00 a.m.

### Study Evaluations

Subjects were required to complete a washout period prior to participation in the study. Subjects who successfully completed the washout period and continued to meet eligibility criteria were randomly assigned in equal number to one of three treatment groups (the results of two the treatment groups are provided in this example); (1) Paliperidone OROS or (2) risperidone immediate release. During the double-blind treatment phase (days 1 to 6), study medication was administered after a standard breakfast. All meals during the study, including the washout and treatment periods, were standardized, consisting of a normal sodium diet with approximately 2,500 Kcal per 24 hours. The subject's total fluid intake was monitored and was not to exceed 3,000 mL per 24 hours. Subjects were to have no more than 3 methylxanthin-containing beverages(e.g. cola, tea coffee) daily. Soft drinks were permitted as long as they did not contain quinine or caffeine and did not increase the permitted total daily calories allowance. Other foods including chocolate, grapefruit, grapefruit juice, Seville oranges and quinine-containing beverages were not allowed. Strenuous physical activity was not permitted during the study.

### Pharmacokinetic Evaluations

At time points during the double-blind treatment period until the end of the study on Day 7, blood samples were obtained for measurement of risperidone, paliperidone and active moiety concentrations in plasma. In each treatment group, a venous blood sample of 5 mL was drawn to obtain about 2 to 3 mL of plasma. The collection schedule was as follows:
On Day 1, immediately before dosing and at 1, 2, 3, 4, 6, 8, 12, 18, and 22 hours postdose:
On Day 2 through Day 5, immediately before dosing and at 2 and 22 hours postdose;
On Day 6, immediately before dosing and at 1, 2, 3, 4, 6, 8, 12, 18, 22, and 24 hours postdose (on Day 7).

Plasma concentrations of risperidone and paliperidone were determined by using a validated liquid chromatography coupled to mass spectrometry/mass spectrometry method.

For the subjects who received IR risperidone, the active moiety concentrations were calculated as the sum of the risperidone and paliperidone concentrations.

### EXAMPLE 5

### Sleep Study

Purpose: Nighttime sleep disturbance and daytime drowsiness are common in patients with schizophrenia, and can be a significant concern for patients that is not always effectively addressed with pharmacologic treatment. Paliperidone extended-release tablet (paliperidone ER) is an investigational psychotropic/antipsychotic agent, with once-daily dosing, that has been shown to be efficacious and well-tolerated in 6-week trials of patients with schizophrenia¹⁻³. Effects of paliperidone ER on changes in sleep architecture (Dataset 1) were studied in patients with schizophrenia-related insomnia, and the clinical importance of these findings was further assessed by patient-rated changes in quality of sleep and daytime drowsiness (Dataset 2) in patients with schizophrenia.

Methods: Data were collected in multicenter, double-blind, randomized and placebo-controlled studies in patients with either stable (Dataset 1) or acute (Dataset 2) schizophrenia. In Dataset 1 (n=42) paliperidone ER 9 mg or placebo were administered daily for 2 weeks. Polysomnography (PSG) variables were assessed at baseline and endpoint. Other sleep measures included Leeds Sleep Evaluation Questionnaire (LSEQ). Dataset 2 (n=1326) was a post-hoc analysis of pooled data from three, 6-week studies¹⁻³ with paliperidone ER 3 mg, 6 mg, 9 mg, 12 mg, 15 mg, or placebo daily. The paliperidone ER capsules were prepared as described in Example 2 and 3 above. Patients dosed with 6 mg were provided with two 3 mg tablets. Patients dosed with 12 mg were provided with one 3 mg and one 9 mg tablet. Patients dosed with 15 mg were provided a 9 mg and two 3 mg tablets. Subjects completed Visual Analogue Scale (VAS) assessments of sleep quality (‘How well have you slept in the past 7 nights?’) and daytime drowsiness (‘How often have you felt drowsy in the past 7 days?’).

Results: Dataset 1 analysis set (n=36) consisted of 67% male, mean±SD age=32.2 y±7.3, baseline mean±SD PANSS total score=62.9±11.2, mean PSG measured latency to persistent sleep of 58.7±45.8 minutes, sleep onset latency of 49.9±41.1 minutes, and a severity of insomnia confirmed by a baseline mean Sleep Efficiency Index (SEI) of 76±13.4%. Treatment with paliperidone ER resulted in clinically robust and statistically significant improvements in sleep, including decreases in latency to persistent sleep (41.9±23.7 minutes) and sleep onset latency (35.9±20.6 minutes). SEI was improved (77.5±15.6%), total sleep time increased (370.4±74.3 minutes), Stage II sleep duration increased (219.3±52.5 minutes), and REM sleep duration increased (80.1±24.6 minutes). Paliperidone ER was without significant effects on slow wave sleep. No statistically significant difference was observed for any change in LSEQ score. The clinical relevance of these findings was supported by Dataset 2 (ITT set: n=1306, 62% male, mean±SD age=38.1 y±10.9, baseline mean±SD PANSS total score=93.5±11.8). All 5 doses of paliperidone ER provided significantly greater improvements in quality of sleep compared with placebo, as rated by VAS assessment (mean±SD baseline/change at endpoint: paliperidone ER 3 mg=56.4±28.8/9.0±34.5; paliperidone ER 6 mg=53.5±30.3/11.1±33.7; paliperidone ER 9 mg=56.5±28.7/11.4±33.0; paliperidone ER 12 mg=55.7±28.0/9.7±33.7; paliperidone ER 15 mg=53.2±29.1/11.3±33.2; placebo=52.4±29.3/0.6±35.9 [p<0.05]). No significant daytime drowsiness was associated with paliperidone ER treatment (mean±SD baseline/change at endpoint: paliperidone ER 3 mg=29.4±26.6/−2.9±28.1; paliperidone ER 6 mg=28.3±25.1/−0.9±27.9; paliperidone ER 9 mg=31.5±27.3/−4.1±32.4; paliperidone ER 12 mg=32.5±26.8/−2.9±31.0; paliperidone ER 15 mg=38.5±30.3/−3.8±34.5; placebo=32.3±27.3/−3.0±30.0 [p<0.05]).

Conclusions: These studies suggest that paliperidone ER treatment resulted in improvements in sleep architecture and sleep continuity and patient-rated sleep quality without producing or exacerbating the daytime drowsiness that is typically often associated with therapies effective for the treatment of insomnia.

We claim:

1. A method for the treatment of a psychiatric patient with excessive daytime sleepiness and/or a sleep disturbance comprising administering a therapeutically effective amount of paliperidone, its pharmaceutically acceptable acid addition salts, enantiomeric forms and esters thereof in a dosage form that provides a relatively low plasma concentration variation of paliperidone to the psychiatric patient in need of treatment.

2. The method of claim 1 wherein the psychiatric patient has excessive daytime sleepiness.

3. The method of claim 1 wherein the psychiatric patient has a sleep disturbance.

4. The method of claim 1 wherein the psychiatric patient has a mental disorder or mental illness selected from the group consisting of schizophrenia, bipolar diseases and other disease states in which psychosis aggressive behavior anxiety or depression is evidenced.

5. The method of claim 4 wherein the psychiatric patient has a schizophrenia type mental disorder or mental illness selected from the group consisting of schizophrenia, schizoaffective disorder and schizophreniform disorders.

6. The method of claim 4 wherein the psychiatric patient has a bipolar mental disorder or mental illness selected from the group consisting of Bipolar I disorder and Bipolar II disorder.

7. The method of claim 1 wherein the psychiatric patient has a mental disorder or mental illness selected from the group consisting of Mild Mental Retardation (317), Moderate Mental Retardation (318.0), Severe Mental Retardation (318.1), Profound Mental Retardation (318.2), Mental Retardation Severity Unspecified (319), Autistic Disorders (299.00), Rett's Disorder (299.80), Childhood Disintegrative Disorders (299.10), Asperger's Disorder (299.80), Pervasive Developmental Disorder Not Otherwise Specified (299.80), Attention-Deficit/Hyperactivity Disorder Combined Type (314.01), Attention-Deficit/Hyperactivity Disorder Predominately Inattentive Type (314.00), Attention-Deficit/Hyperactivity Disorder Predominately Hyperactive-Impulsive Type (314.01), Attention-Deficit/Hyperactivity Disorder NOS (314.9) Conduct Disorder (Childhood-Onset and Adolescent Type 312.8) Oppositional Defiant Disorder (313.81), Disruptive Behavior Disorder Not Otherwise Specified (312.9), Solitary Aggressive Type (312.00), Conduct Disorder, Undifferentiated Type (312.90), Tourette's Disorder (307.23), Chronic Motor Or Vocal Tic Disorder (307.22), Transient Tic Disorder (307.21), Tic Disorder NOS (307.20), Alcohol Intoxication Delirium (291.0), Alcohol Withdrawal Delirium (291.0), Alcohol-Induced Persisting Dementia (291.2), Alcohol-Induced Psychotic Disorder with Delusions (291.5), Alcohol-Induced Psychotic Disorder with Hallucinations (291.3), Amphetamine or Similarly Acting Sympathomimetic Intoxication (292.89), Amphetamine or Similarly Acting Sympathomimetic Delirium (292.81), Amphetamine or Similarly Acting Sympathomimetic Induced Psychotic with Delusional (292.11), Amphetamine or Similarly Acting Sympathomimetic Induced Psychotic with Hallucinations (292.12), Cannabis-Induced Psychotic Disorder with Delusions (292.11), Cannabis-Induced Psychotic Disorder with Hallucinations (292.12), Cocaine Intoxication (292.89), Cocaine Intoxication Delirium (292.81), Cocaine-Induced Psychotic Disorder with Delusions (292.11), Cocaine-Induced Psychotic Disorder with Hallucinations (292.12), Halluciogen Intoxication (292.89), Hallucinogen Intoxication Delirium (292.81), Hallucinogen-Induced Psychotic disorder with Delusions (292.11), Hallucinogen-Induced Psychotic disorder with Delusions (292.12), Hallucinogen-Induced Mood Disorder (292.84), Hallucinogen-Induced Anxiety Disorder (292.89), Hallucinogen-Related Disorder Not Otherwise Specified (292.9), Inhalant Intoxication (292.89), Inhalant Intoxication Delirium (292.81), Inhalant-Induced Persisting Dementia (292.82), Inhalant-Induced Psychotic Disorder with Delusions (292.11), Inhalant-Induced Psychotic with Hallucinations (292.12), Inhalant-Induced Mood Disorder (292.89), Inhalant-Induced Anxiety Disorder (292.89), Inhalant-Related Disorder Not Otherwise Specified (292.9), Opioid Intoxication Delirium (292.81), Opioid-Induced Psychotic Disorder with Delusions (292.11), Opioid Intoxication Delirium (292.81), Opioid-Induced Psychotic Disorder with Hallucinations (292.12), Opioid-Induced Mood Disorder (292.84), Phencyclidine (PCP) or Similarly Acting Arylcyclohexylamine Intoxication (292.89), Phencyclidine (PCP) or Similarly Acting Arylcyclohexylamine Intoxication Delirium (292.81), Phencyclidine (PCP) or Similarly Acting Arylcyclohexylamine Induced Psychotic Disorder with Delusions (292.11), Phencyclidine (PCP) or Similarly Acting Arylcyclohexylamine Induced Psychotic Disorder with Hallucinations (292.12), Phencyclidine (PCP) or Similarly Acting Arylcyclohexylamine Mood Disorder (292.84), Phencyclidine (PCP) or Similarly Acting Arylcyclohexylamine Induced Anxiety Disorder (292.89), Phencyclidine (PCP) or Similarly Acting Arylcyclohexylamine Related Disorder Not Otherwise Specified (292.9), Sedative, Hypnotic or Anxiolytic Intoxication (292.89), Sedation, Hypnotic or Anxiolytic Intoxication Delirium (292.81), Sedation, Hypnotic or Anxiolytic Withdrawal Delirium (292.81), Sedation, Hypnotic or Anxiolytic Induced Persisting Dementia (292.82), Sedation, Hypnotic or Anxiolytic-Induced Psychotic Disorder with Delusions (292.11), Sedation, Hypnotic or Anxiolytic-Induced Psychotic Disorder with Hallucinations (292.12), Sedation, Hypnotic or Anxiolytic-Induced Mood Disorder (292.84), Sedation, Hypnotic or Anxiolytic-Induced Anxiety Disorder (292.89), Other (or Unknown) Substance Intoxication (292.89), Other (or Unknown) Substance-Induced Delirium (292.81), Other (or Unknown) Substance-Induced Persisting Dementia (292.82), Other (or Unknown) Substance-Induced Psychotic Disorder with Delusions (292.11), Other (or Unknown) Substance-Induced Psychotic Disorder with Hallucinations (292.12), Other (or Unknown) Substance-Induced Mood Disorder (292.84), Other (or Unknown) Substance-Induced Anxiety Disorder (292.89), Other (or Unknown) Substance Disorder Not Otherwise Specified (292.9), Obsessive Compulsive Disorder (300.3), Post-traumatic Stress Disorder (309.81), Generalized Anxiety Disorder (300.02), Anxiety Disorder Not Otherwise Specified (300.00), Body Dysmorphic Disorder (300.7), Hypochondriasis (or Hypochondriacal Neurosis) (300.7), Somatization Disorder (300.81), Undifferentiated Somatoform Disorder (300.81), Somatoform Disorder Not Otherwise Specified (300.81), Intermittent Explosive Disorder (312.34), Kleptomania (312.32), Pathological Gambling (312.31), Pyromania (312.33), Trichotillomania (312.39), and Impulse Control Disorder NOS (312.30), Schizophrenia, Paranoid Type, (295.30), Schizophrenia, Disorganized (295.10), Schizophrenia, Catatonic Type, (295.20), Schizophrenia, Undifferentiated Type (295.90), Schizophrenia, Residual Type (295.60), Schizophreniform Disorder (295.40), Schizoaffective Disorder (295.70), Delusional Disorder (297.1), Brief Psychotic Disorder (298.8), Shared Psychotic Disorder (297.3), Psychotic Disorder Due to a General Medical Condition with Delusions (293.81), Psychotic Disorder Due to a General Medical Condition with Hallucinations (293.82), Psychotic Disorders Not Otherwise Specified (298.9), Major Depression, Single Episode, Severe, without Psychotic Features (296.23), Major Depression, Recurrent, Severe, without Psychotic Features (296.33), Bipolar Disorder, Mixed, Severe, without Psychotic Features (296.63), Bipolar Disorder, Mixed, Severe, with Psychotic Features (296.64), Bipolar Disorder, Manic, Severe, without Psychotic Features (296.43), Bipolar Disorder, Manic, Severe, with Psychotic Features (296.44), Bipolar Disorder, Depressed, Severe, without Psychotic Features (296.53), Bipolar Disorder, Depressed, Severe, with Psychotic Features (296.54), Bipolar II Disorder (296.89), Bipolar Disorder Not Otherwise Specified (296.80), Personality Disorders, Paranoid (301.0), Personality Disorders, Schizoid (301.20), Personality Disorders, Schizotypal (301.22), Personality Disorders, Antisocial (301.7), and Personality Disorders, and Borderline (301.83).

8. The method of claim 3 wherein the mental illness is selected from the group consisting of Post traumatic Stress Disorder (309.81); Schizophrenia, Paranoid Type, (295.30); Schizophrenia, Disorganized (295.10); Schizophrenia, Catatonic Type, (295.20); Schizophrenia, Undifferentiated Type (295.90); Schizophrenia, Residual Type (295.60); Schizophreniform Disorder (295.40); Schizoaffective Disorder (295.70); Delusional Disorder (297.1); Brief Psychotic Disorder (298.8); Shared Psychotic Disorder (297.3); Psychotic Disorder Due to a General Medical Condition with Delusions (293.81); Psychotic Disorder Due to a General Medical Condition with Hallucinations (293.82); Psychotic Disorders Not Otherwise Specified (298.9); Major Depression, Single Episode, Severe, without Psychotic Features (296.23); Major Depression, Recurrent, Severe, without Psychotic Features (296.33); Bipolar Disorder, Mixed, Severe, without Psychotic Features (296.63); Bipolar Disorder, Mixed, Severe, with Psychotic Features (296.64); Bipolar Disorder, Manic, Severe, without Psychotic Features (296.43); Bipolar Disorder, Manic, Severe, with Psychotic Features (296.44); Bipolar Disorder, Depressed, Severe, without Psychotic Features (296.53); Bipolar Disorder, Depressed, Severe, with Psychotic Features (296.54); Bipolar II Disorder (296.89); Bipolar Disorder Not Otherwise Specified (296.80); Personality Disorders, Paranoid (301.0); Personality Disorders, Schizoid (301.20); Personality Disorders, Schizotypal (301.22); Personality Disorders, Antisocial (301.7); and Personality Disorders, and Borderline (301.83).

9. The method of claim 1 wherein the low plasma concentration variation comprises a change in plasma concentration (Cmax-Cmin/Cmax) of less than about 30 percent,

10. The method of claim 1 wherein the low plasma concentration variation comprises a change in plasma concentration (Cmax-Cmin/Cmax) of from about 10 percent to about 30 percent,

11. The method of claim 1 wherein the low plasma concentration variation comprises a change in plasma concentration (Cmax-Cmin/Cmax) of from about 15 percent to about 25 percent.

12. A method for the treatment of a psychiatric patient having a mental illness selected from the group consisting of Post traumatic Stress Disorder (309.81); Schizophrenia, Paranoid Type, (295.30); Schizophrenia, Disorganized (295.10); Schizophrenia, Catatonic Type, (295.20); Schizophrenia, Undifferentiated Type (295.90); Schizophrenia, Residual Type (295.60); Schizophreniform Disorder (295.40); Schizoaffective Disorder (295.70); Delusional Disorder (297.1); Brief Psychotic Disorder (298.8); Shared Psychotic Disorder (297.3); Psychotic Disorder Due to a General Medical Condition with Delusions (293.81); Psychotic Disorder Due to a General Medical Condition with Hallucinations (293.82); Psychotic Disorders Not Otherwise Specified (298.9); Major Depression, Single Episode, Severe, without Psychotic Features (296.23); Major Depression, Recurrent, Severe, without Psychotic Features (296.33); Bipolar Disorder, Mixed, Severe, without Psychotic Features (296.63); Bipolar Disorder, Mixed, Severe, with Psychotic Features (296.64); Bipolar Disorder, Manic, Severe, without Psychotic Features (296.43); Bipolar Disorder, Manic, Severe, with Psychotic Features (296.44); Bipolar Disorder, Depressed, Severe, without Psychotic Features (296.53); Bipolar Disorder, Depressed, Severe, with Psychotic Features (296.54); Bipolar II Disorder (296.89); Bipolar Disorder Not Otherwise Specified (296.80); Personality Disorders, Paranoid (301.0); Personality Disorders, Schizoid (301.20); Personality Disorders, Schizotypal (301.22); Personality Disorders, Antisocial (301.7); and Personality Disorders, and Borderline (301.83)with excessive daytime sleepiness and/or a sleep disturbance comprising administering a therapeutically effective amount of paliperidone, its pharmaceutically acceptable acid addition salts, enantiomeric forms and esters thereof in a dosage form that provides a relatively low plasma concentration variation of paliperidone to the psychiatric patient in need of treatment.

13. The method of claim 12 wherein the low plasma concentration variation comprises a change in plasma concentration (Cmax-Cmin/Cmax) of less than about 30 percent,

14. The method of claim 12 wherein the low plasma concentration variation comprises a change in plasma concentration (Cmax-Cmin/Cmax) of from about 10 percent to about 30 percent,

15. The method of claim 12 wherein the low plasma concentration variation comprises a change in plasma concentration (Cmax-Cmin/Cmax) of from about 15 percent to about 25 percent.

16. The method of claim 12 wherein the psychiatric patient has excessive daytime sleepiness.

17. The method of claim 12 wherein the psychiatric patient has a sleep disturbance.
